(19) United States
(12) Patent Application Publication
(10) Pub. No.: US 2006/0020225 A1
(43) Pub. Date: Jan. 26, 2006

(54) Wireless urodynamic monitoring system with automated voiding diary

(76) Inventors: Martin T. Gerber, Maple Grove, MN (US); William D. Weimer, Plymouth, MN (US)

Correspondence Address: SHUMAKER & SIEFFERT, P. A., 8425 SEASONS PARKWAY, SUITE 105, ST. PAUL, MN, 55125, US

(21) Appl. No.: 10/978,233

(22) Filed: Oct. 29, 2004

Related U.S. Application Data
(60) Provisional application No. 60/589,442, filed on Jul. 20, 2004.
(60) Provisional application No. 60/589,542, filed on Jul. 20, 2004.

(51) Int. Cl. A61B 5/00

(52) U.S. Cl. 600/561; 600/549

(57) ABSTRACT

The disclosure describes a wireless urodynamic monitoring system with an automated voiding diary feature. The system senses and records urodynamic information in response to a user command or in response to detection of the onset of a voiding event. The urodynamic information obtained over a series of voiding events forms an automated voiding diary that is useful in diagnosis of urological disorders. An implantable monitor obtains the urodynamic information and either records the information locally or transmits the information to an external controller via a wireless telemetry link. In some embodiments, the external controller may include a loop recorder for recording urodynamic information obtained by the implantable monitor over an extended period of time.

This application claims the benefit of U.S. provisional application No. 60/589,442, filed Jul. 20, 2004, and U.S. provisional application No. 60/589,542, filed Jul. 20, 2004, the entire content of each of which is incorporated herein by reference.

### FIELD OF THE INVENTION

The invention relates to medical devices and, more particularly, devices for monitoring urodynamic conditions.

### BACKGROUND

Many people suffer from involuntary urine leakage, i.e., urinary incontinence. Others may suffer from blocked or restricted urine flow. Other urinary disorders include frequent urination, sudden urges to urinate, problems starting a urine stream, painful urination, problems emptying the bladder completely, and recurrent urinary tract infections. A physician uses a urodynamic test to study how a patient stores and releases urine. During the test, the physician obtains urodynamic information based on one or more physiological conditions within the urinary tract.

Different muscles, nerves, organs and conduits within the urinary tract cooperate to collect, store and release urine. A variety of disorders may compromise the urinary tract performance and contribute to incontinence or restricted flow. Many of the disorders may be associated with aging, injury or illness. For example, aging can often result in weakened sphincter muscles, which cause incontinence, or weakened bladder muscles, which prevent complete emptying. Some patients also may suffer from nerve disorders that prevent proper triggering and operation of the bladder or sphincter muscles.

Urodynamic sensing can reveal how well the bladder and sphincter muscles perform, and may help identify the causes of various urinary tract disorders. Urodynamic sensing can take the form of simple observation or precise measurement using monitors that sense physiological conditions such as urine pressure, flow, velocity, volume, and the like. Some monitors sense the occurrence and force of bladder contractions to identify abnormal bladder function. Other monitors may determine a volume of urine remaining in the bladder following urination. Hence, urodynamic sensing may focus on the ability of the bladder to empty steadily and completely.

Urodynamic testing ordinarily requires catheterization of the patient in order to place a monitor within the bladder or urethra. For this reason, urodynamic testing typically takes place within a clinical setting. In some cases, the presence of a catheter can disrupt the normal physiological function of the urinary tract. Although ambulatory catheterization is possible, it can be uncomfortable and may result in measurements that are not be representative of normal physiological function. In addition, the urinary catheter can be uncomfortable for the patient.

Various urodynamic testing systems are described in U.S. Pat. No. 4,873,990 to Holmes et al., U.S. Pat. No. 5,331,548 to Rollema et al., and U.S. Pat. No. 6,454,720 to Clerc et al. Siwapomsathain et al. describe a bladder monitor with wireless telemetry in Siwapornsathain et al., “A Telemetry and Sensor Platform for Ambulatory Urodynamics,” Proceedings of the 2nd Annual International IEEE-EMBS Special Topic Conference on Microtechnologies in Medicine & Biology, Madison, Wis., 2002. J. Coosemans et al. describe an implantable bladder pressure monitor with wireless telemetry in “Datalogger for Bladder Pressure Monitoring With Wireless Power and Data Transmission,” Katholieke Universiteit Leuven, Department ESAT-MICAS, Belgium, Belgian Day on Biomedical Engineering, 2003.

Table 1 below lists documents that disclose various techniques for urodynamic testing.

TABLE 1

| | Inventors/ | |
|---|---|---|
| Patent Number | Author | Title |
| 4,873,990 | Holmes et al. | Circumferential Pressure Probe |
| 5,331,548 | Rollema et al. | Method and system for on-line measure- |
| | | ment, storage, retrieval and analysis |
| | | of urodynamical data |
| 6,454,720 | Clerc et al. | System for measuring physical para- |
| | | meters with a medical probe |
| Not applicable | J. Coosemans | Datalogger for Bladder Pressure Moni- |
| | et al. | toring With Wireless Power and Data |
| | | Transmission |
| Not Applicable | Siwapornsa- | A Telemetry and Sensor Platform for |
| | thain et al. | Ambulatory Urodynamics |

All documents listed in Table 1 above are hereby incorporated by reference herein in their respective entireties. As those of ordinary skill in the art will appreciate readily upon reading the Summary of the Invention, Detailed Description of the Preferred Embodiments and claims set forth below, many of the devices and methods disclosed in the patents of Table 1 may be modified advantageously by using the techniques of the present invention.

### SUMMARY OF THE INVENTION

In general, the invention is directed to a wireless urodynamic monitoring system with an automated voiding diary feature. The system senses and records urodynamic information in response to a user command or in response to detection of the onset of a voiding event. The urodynamic information obtained over a series of voiding events forms an automated voiding diary that is useful in diagnosis of urological disorders. An implantable monitor obtains the urodynamic information and either records the information locally or transmits the information to an external controller via a wireless telemetry link. In some embodiments, the external controller may include a loop recorder for recording urodynamic information obtained by the implantable monitor over an extended period of time.

Various embodiments of the present invention provide solutions to one or more problems existing in the prior art with respect to prior art systems for urodynamic monitoring. These problems include the general inconvenience and discomfort experienced by the patient during the use of catheter-based urodynamic monitoring systems. Other problems relate to potential inconsistencies involved in the manual preparation of voiding diaries by individual patients. Further problems relate to the difficulty in the transfer and analysis of voiding diary information. Such problems can limit the collection and analysis of useful urodynamic information, and undermine the integrity and diagnostic efficacy of the information.

Various embodiments of the present invention are capable of solving at least one of the foregoing problems. When embodied in a system or method for wireless urodynamic monitoring, the invention includes features that facilitate automated formulation of a voiding diary. A wireless urodynamic monitoring system, in accordance with the invention, includes a wireless implantable monitor and an external controller. The implantable monitor and external controller can accompany the patient throughout a routine of normal daily activities.

The implantable monitor is configured for indwelling urodynamic monitoring within the bladder or urethra. The wireless implantable monitor obtains urodynamic information in response to a voiding event activation command entered into the external controller or automated detection of the onset of a voiding event. The monitor may be configured to transmit the information to the external controller. In this manner, the monitoring system selectively records urodynamic information obtained at the time of a voiding event.

In comparison to known techniques for monitoring urodynamic parameters, various embodiments of the invention may provide one or more advantages. For example, the invention facilitates the automated acquisition, transfer and analysis of voiding diary information. In addition, the use of a wireless urodynamic monitor can reduce patient inconvenience and discomfort, and facilitate urodynamic monitoring as the patient goes about his daily routine. Also, by acquiring urodynamic information automatically in response to either patient input or detection of the onset of a voiding event, a wireless monitoring system supports convenient recording of urodynamic information that is particularly relevant and useful in diagnosis of urological disorders. Automated formulation of a voiding diary also avoids potential inconsistencies in manual voiding diaries, which may rely on individual patient discipline.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a schematic diagram illustrating a wireless urodynamic monitor and an external controller shown in conjunction with the bladder and urethra of a patient.

FIG. 2 is a functional block diagram illustrating a wireless urodynamic monitor with a telemetry interface for communication with an external controller.

FIG. 3 is a functional block diagram illustrating an external controller with a telemetry interface for wireless communication with an implanted urodynamic monitor.

FIG. 4 is flow diagram illustrating operation of a urodynamic monitor and external controller in response to a voiding activation input from a patient.

FIG. 5 is flow diagram illustrating operation of a urodynamic monitor and external controller in response to a sensed voiding event.

FIG. 6 is a functional block diagram illustrating a network for communication of information obtained by wireless urodynamic monitors.

FIG. 7 is a cross-sectional side view of a wireless urodynamic monitor attached to a tissue site within the bladder or urethra.

FIG. 8 is a schematic diagram illustrating deployment of the monitor of FIG. 7 within a patient's urinary tract with an endoscopic delivery device.

FIG. 9 is a cross-sectional side view of the wireless urodynamic monitor and distal end of the endoscopic delivery device of FIGS. 7 and 8.

FIG. 10 is a side view of a monitor with a fixation structure in the form of an expandable frame.

FIG. 11 is a conceptual diagram of an external controller equipped to record and present information obtained from an implantable urodynamic monitor.

FIG. 12 is another conceptual diagram of the external controller of FIG. 11.

### DESCRIPTION OF THE PREFERRED EMBODIMENTS

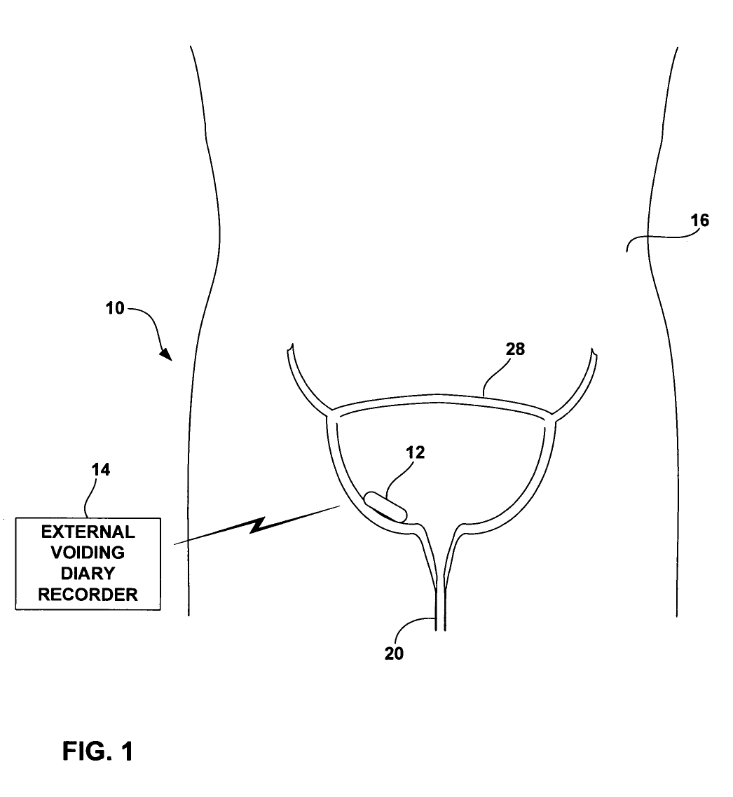

FIG. 1 is a schematic diagram illustrating a wireless urodynamic monitoring system 10 including an implanted urodynamic monitor 12 and an external controller 14 shown in conjunction with a patient 16. System 10 collects and records urodynamic information in response to a voiding event activation command. The voiding event activation command may be entered into external controller 14 by patient 16 to indicate the onset of a voiding event. Alternatively, the voiding event activation command may be generated automatically within monitor 12 or external controller 14 upon detection of urodynamic information indicating the onset of a voiding event. Implantable monitor 12 and external controller 14 cooperate to form an automated voiding diary containing urodynamic information obtained over a series of voiding events.

Urodynamic monitor 12 may be implanted within a patient bladder 18 or urethra 20 forming part of the patient's urinary tract. In FIG. 1, urodynamic monitor 12 is shown at a target location within bladder 18, but alternatively may be implanted within urethra 20. In some embodiments, multiple urinary tract monitors 18 may be placed within the urinary tract. Monitor 12 may be placed at a target location within the urinary tract by endoscopic delivery, e.g., using a catheter, cystoscope, endoscope, or the like, as will be described in greater detail. Monitor 12 may be implanted temporarily or chronically.

The target location may be within bladder 18 or within urethra 20. As will be described in greater detail, monitor 12 may include any of a variety of fixation structures to securely position the monitor at a target tissue location within the urinary tract. Upon fixation of monitor 12, the endoscopic delivery device may be withdrawn from the urinary tract of patient 16. In this manner, monitor 12 can remain in a desired position for an extended period of time, and accompany patient 16 outside the clinic or hospital and throughout a routine of daily activities.

Urodynarnic information, as used herein, generally refers to physiological information characterizing or relating to the function of the bladder 18, urethra 20, or other segments of a patient's urinary tract in storing, releasing, and passing urine. The urodynamic information may include urine pressure, flow, velocity, temperature, impedance, or the like, as well as contractile activity of bladder 18. A voiding event, as used herein, generally refers to a time at which a patient attempts to void urine from the bladder, feels discomfort in the bladder or urethra, or experiences involuntary urine leakage or other urinary incontinence symptoms. Each type of voiding event can provide important urodynamic information for evaluation of urological health. A voiding diary, in general, refers to a set of urodynamic information associated with particular voiding events.

Patient 16 may activate urodynamic monitor 12 to collect urodynamic information during or proximate to a urinary voiding event. For example, patient 16 may activate urodynamic monitor 12 by entering a voiding event activation command into external controller 14 to indicate the onset of a voiding event, such as an attempt to void urine from bladder 18. In response, external controller 14 transmits a wireless command to urodynamic monitor 12, which then captures one or more measurements of urodynamic parameters within bladder 18 or urethra 20 to form a set of urodynamic information. Alternatively, in some embodiments, urodynamic monitor 12 may automatically detect the onset of a voiding event, and automatically trigger the collection of urodynamic information.

In some embodiments, urodynamic monitor 12 transmits the urodynamic information to external controller 14, e.g., as the information is obtained. External controller 14 generates a voiding diary that contains a record of voiding events and urodynamic information measured by urodynamic monitor 12 during the voiding events. In this manner, external controller 14 selectively records relevant information obtained at the time a patient experiences a voiding event, such as an attempt to void urine from the bladder. Hence, there is no need for the patient 16 to make a manual voiding diary. Also, the automated voiding diary contains objective urodynamic information, rather than subjective impressions from the patient.

In other embodiments, rather than immediately transmitting the urodynamic information to external controller 14, monitor 12 may initially store the information internally for subsequent wireless transmission to external controller 14 or another device. Hence, in some embodiments, the urodynamic information may be stored within monitor 12, and later transmitted to external controller 14 upon interrogation of the monitor. Interrogation may be initiated by the patient 16 by entering a command into external controller 14. In further embodiments, monitor 12 may store the information internally on a persistent basis for later retrieval by external controller 14, or upon explantation of the monitor.

Urodynamic monitor 12 is configured for indwelling urodynamic testing within the bladder 18 or urethra 20. In this manner, monitor 12 can accompany patient 16 throughout a routine of normal daily activities. In some embodiments, multiple urodynamic monitors 12 may be implanted within the urinary tract of patient 16. External controller 14 may take the form of a handheld, external recorder that is carried by patient 16.

Urodynamic monitor 12 may obtain a variety of urodynamic information relating to physiological conditions within the bladder 18 or urethra 20 during a voiding event. For example, the physiological conditions may include one or more urodynamic conditions such as urine pressure, urine volume, urine flow, urine pH, temperature, bladder contraction, or urinary sphincter contraction. The urodynamic information may represent averages, trends, or instantaneous measurements, as well as information representing specific events during the course of voiding. As a further example, implanted monitor 12 may measure and record an indication of the residual volume of urine following a voiding event. Residual volume is another important parameter in urodynamic studies to measure the effectiveness of the voiding bladder condition. In some embodiments, urodynamic monitor 12 and external controller 14 may be configured as loop recorders to overwrite their respective memories with new information as the memory becomes full.

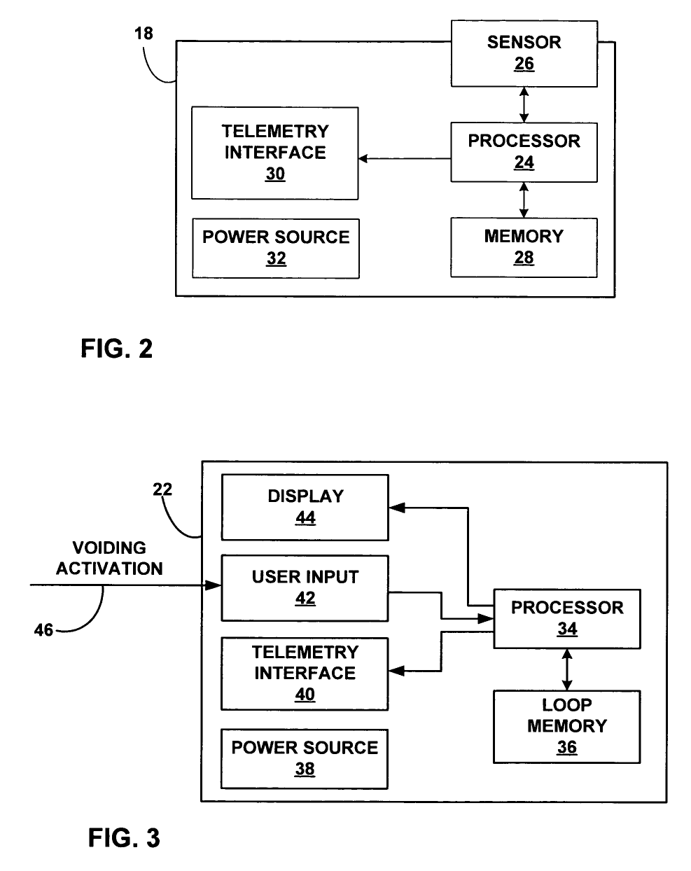

FIG. 2 is a functional block diagram illustrating implantable urodynamic monitor 12 of FIG. 1. In the example of FIG. 2, monitor 12 includes a processor 24, a sensor 26, memory 28, wireless telemetry interface 30, and a power source 32. Monitor 12 also may include an internal clock to track date and time of voiding events. In accordance with the invention, monitor 12 obtains urodynamic information via sensor 26 in response to a voiding event activation command, which may be initiated by a user or generated automatically upon detection of the onset of a voiding event.

Monitor 12 may be entirely self-contained, self-powered and integrated within a common housing using miniaturized integrated circuitry available to those skilled in the art. In some embodiments, for example, monitor 12 may be constructed in a manner similar to the monitors described in U.S. patent application Ser. No. 10/833,776, to Mark Christopherson and Warren Starkebaum, filed Apr. 28, 2004, entitled “Implantable Urinary Tract Monitor,” the entire content of which is incorporated herein by reference.

Power source 32 may take the form of a small battery. An external source of inductively coupled power may be used, in some embodiments, to power some features of monitor 12. For example, monitor 12 may include an inductive power interface for transcutaneous inductive power transfer to power higher energy functions such as telemetry. However, monitor 12 typically will include a small battery cell within the monitor housing. Alternatively, monitor 12 may include an inductive power interface in lieu of a battery.

Telemetry interface 30 permits wireless communication with external controller 14 for wireless transmission of information obtained by sensor 26, as well as wireless reception of a voiding event activation command directing monitor 12 to collect physiological information during an attempt to void urine from the bladder. As a further alternative, the voiding event activation command may be applied by patient 16 in the form of a magnet swiped in proximity to monitor 12, in which case the monitor will include appropriate sensing circuitry to detect the magnet. Accordingly, an optional magnet detector 33 is shown in FIG. 3.

Processor 24 controls telemetry interface 30 and handles processing and storage of information obtained by sensor 26. Processor 24 controls operation of monitor 12 and may include one or more microprocessors, digital signal processors (DSPs), application-specific integrated circuits (ASICs), field-programmable gate arrays (FPGAs), or other equivalent logic circuitry. Memory 28 may include any magnetic, electronic, or optical media, such as random access memory (RAM), read-only memory (ROM), electronically-erasable programmable ROM (EEPROM), flash memory, or the like, or a combination thereof. Memory 28 may store program instructions that, when executed by processor 24, cause the controller to perform the functions ascribed to it herein. For example, memory 28 may store instructions for processor 24 to execute in support of control of wireless telemetry interface 30 and control of, and processing of information obtained by, sensor 26. Memory 28 may include separate memories for storage of instructions and urodynamic information.

Telemetry interface 30 may include a wireless radio frequency (RF) transmitter and receiver to permit bi-directional communication between monitor 12 and external controller 14. In this manner, external controller 14 may transmit commands to urodynamic monitor 12 for collection of urodynamic information or collection of information stored in memory 28, and receive status and operational information from the monitor. Telemetry interface 30 includes an antenna, which may take a variety of forms. For example, the antenna may be formed by a conductive coil or wire embedded in a housing associated with monitor 12. Alternatively, the antenna may be mounted on a circuit board carrying other components of monitor 12, or take the form of a circuit trace on the circuit board.

Battery power source 32 may take the form of a battery and power generation circuitry. Urodynamic monitor 12 typically may be used for a few days or weeks, and therefore may not require substantial battery resources. Accordingly, the battery within battery power source 32 may be very small. An example of a suitable battery is the Energizer 337 silver oxide cell, available from the Eveready Battery Company, of St. Louis, Mo., USA. The Energizer 337 battery is disc-shaped, and has a diameter of 4.88 mm and thickness of 1.65 mm. Another example battery is the QL003I3 milliamp cylindrical battery from Quallion, LLC, of Sylmar, Calif., USA, which has a diameter of approximately 2.9 mm and a length of approximately 13.0 mm.

In further embodiments, battery power source 32 may be rechargeable via electromagnetic induction or ultrasonic energy transmission, and includes an appropriate circuit for recovering transcutaneously received energy. For example, battery power source 32 may include a secondary coil and a rectifier circuit for inductive energy transfer. In still other embodiments, battery power source 32 may not include any storage element, and monitor 12 may be fully powered via transcutaneous inductive energy transfer, which may be provided by external receiver 14.

Sensor 26 may be selected for any of a variety of urodynamic testing applications, and may include appropriate signal processing circuitry such as amplifier, filter, driver, and analog-to-digital conversion circuitry for presentation of sensed information to processor 24. For urodynamic testing, sensor 26 may take the form of a pressure, flow, velocity, volume, temperature, impedance, or contractile force sensor.

Pressure, contractile force or other measurements may be used to detect bladder or urinary sphincter functions in order to automatically detect the onset of an attempt to void urine from bladder 18. For example, an elevated pressure or force, or change in pressure or force, may indicate a contraction of the bladder muscle, which may be used to generate a voiding event activation command to trigger collection of urodynamic information for recording in a voiding diary. As a further example, if monitor 12 is implanted within urethra 20, the voiding event may be detected by detecting the presence of urine in the urethra, e.g., by flow, pressure, temperature, or impedance sensing. Accordingly, in some embodiments, the use of two monitors 12 may be desirable, e.g., one in bladder 18 and one in urethra 20. Hence, for automated detection, processor 24 may periodically sample and monitor the output of sensor 26. In some embodiments, monitor 12 may transmit the periodically sampled information to external controller 14, which then analyzes the information to detect a voiding event.

For urodynamic testing, sensor 26 may have a structure similar to sensors conventionally used for catheter-based urodynamic testing. For pressure measurements, for example, sensor 26 may include one or more diaphragm sensors, strain gauge sensors, capacitive sensors, piezoelectric sensors, or other sensors used in conventional catheter-based urodynamic testing to sense pressure. For bladder emptying, sensor 26 may include a conductive sensor to sense the presence of urine within the lower region of the bladder 18.

For flow measurements, sensor 26 may comprise a pulsed Doppler ultrasonic sensor, or a laser Doppler flow sensor. Doppler shifting of the frequency of the reflected energy indicates the velocity of the fluid flow passing over a surface of sensor 26. Consequently, in some embodiments, monitor 12 may include circuitry, such as a quadrature phase detector, in order to enable the monitor to distinguish the direction of the flow of fluid in addition to its velocity.

As a further example, sensor 26 may include any one or more thermal-convection velocity sensors. A thermal-convection velocity sensor may include a heating element upstream of a thermistor to heat urine within the urethra 16 such that flow rate may be measured according to the temperature of the heated fluid when it arrives at the thermistor. In other embodiments, flow rate may be determined from the output of a concentration or temperature sensor using Fick's techniques.

In some embodiments, sensor 26 may include multiple sensors of a given type, as well as multiple types of sensors, e.g., pressure, flow, bladder emptying, or the like. Accordingly, the urodynamic information obtained by monitor 12 may then include different types of physiological parameters associated with a voiding event. Alternatively, multiple monitors 12 may be deployed within bladder 18 or urethra. In this case, each monitor 12 may be configured with a different type or set of sensors 26 to collect a variety of different urodynamic parameters during a voiding event.

In response to a voiding event activation command, monitor 12 obtains urodynamic information from sensor 26 and either records the information in internal memory 28 carried by the monitor, or transmits the information directly to external controller 14 by wireless telemetry. In some embodiments, monitor 12 also may be capable of continuously or periodically performing urodynamic testing over an extended period of time, encompassing voiding events and measurements between voiding events.

By selectively obtaining urodynamic information associated with voiding events, however, monitor 12 is configured to support the generation of an automatic urinary voiding diary containing urodynamic information obtained over a series of voiding attempts. Again, in response to the voiding event activation command, monitor 12 commences sensing of one or more physiological conditions within bladder 18 or urethra 20, and either records pertinent information internally, transmits the information directly to external controller 14, or temporarily buffers and then transmits the information to external controller 14, either directly or in response to an interrogation request by the external controller.

In embodiments in which information is stored internally within monitor 12, the monitor may include functionality similar to the existing Medtronic Reveal™ implantable loop recorder, manufactured by Medtronic, Inc. of Minneapolis, Minn. The Reveal™ implantable loop recorder samples and records one or more segments of far field EGM or subcutaneous ECG signals.

Aspects of the Reveal™ loop recorder are disclosed in commonly assigned PCT publication WO98/02209, the entire content of which is incorporated herein by reference. In accordance with the invention, monitor 12 may be adapted to sample and record one or more segments of information pertaining to physiological conditions within the urinary tract. In some embodiments, the memory capacity of memory 28 in monitor 12 may be limited, and so the segments of information that are stored in memory can be written over with new information when the patient triggers storage and the memory is full.

The most recently stored segment or segments of information may be transmitted via uplink telemetry transmission from monitor 12 to external controller 14 on a continuous or periodic basis, or when a memory interrogation telemetry session is initiated by a medical care provider using the external controller. In other embodiments, external controller 14 may be configured to avoid overwriting, and instead retain previously sensed information when the memory with the external controller is full.

FIG. 3 is a functional block diagram illustrating external controller 14 for communication with urodynamic monitor 18 of FIG. 2. In the example of FIG. 3, external controller 14 includes a processor 34, memory 36, power source 38, telemetry interface 40, user input 42 and display 44. Memory 36 stores instructions for execution by processor 34. In addition, memory 36 stores information received from monitor 12 incident to a voiding event, thereby forming an automated voiding diary for review by a physician. Memory 36 may include separate memories for storage of instructions and urodynamic information.

Processor 34 controls telemetry interface 40 to obtain urodynamic information from monitor 18. Processor 34 also may control telemetry interface 40 to receive information from monitor 18 on a substantially continuous basis, at periodic intervals, or only upon receipt of a user activation command. Hence, external controller 14 may obtain on ongoing, up-to-date indication of the physiological conditions sensed by monitor 12. More particularly, however, external controller 14 is configured to respond to a voiding event activation command 46 entered by patient 16 via user input device 42. In response to the voiding event activation command 46, external controller 14 generates an activation control signal and transmits the control signal to monitor 12 via telemetry interface 40.

Urodynamic monitor 12 may be configured to collect information for a specified period of time following receipt of an activation control signal from external controller 14, e.g., for a period of several minutes. Alternatively, external controller 14 may receive a voiding event deactivation command (not shown) from the user. In this case, external controller 14 transmits a deactivation control signal to urodynamic monitor 12. Accordingly, monitor 12 may be configured to collect urodynamic information from the time an activation control signal is received to the time a deactivation control signal is received.

External controller 14 permits a user to receive urodynamic information obtained by a sensor carried by monitor 12 during the course of a voiding event. In addition, external controller 14 may process and record information obtained from monitor 12, and present the information to a user via display 44 or other output media. In some embodiments, the information may include one or more advisories with respect to the presence or level of a urodynamic parameter. In addition, the recorded information may be transmitted from external device 14 to other external devices for presentation, archival or further analysis.

Advantageously, the recorder functionality of the external controller 14 serves to build a voiding diary. In particular, by permitting a patient 16 to activate sensing and recording coincident with a voiding event, external controller 14 is able to compile information specifically associated with one or more voiding events over an observation period. Consequently, a physician can view a more selective set of urodynamic information, which may be very useful in diagnosing symptoms such as incontinence, pain, or the like. In particular, a physician can parse through multiple entries in the voiding diary to identify changes in physiological conditions over time, or at different times of the day or night.

Wireless telemetry may be accomplished by radio frequency (RF) communication or proximal inductive interaction of external controller 14 with monitor 12. Alternatively, telemetry interfaces 30, 40 may be configured for monitor 12 and external controller 14 to support radio frequency (RF) communication with a sufficiently strong signal such that proximate interaction is not required. In addition to an RF or inductive telemetry interface 40, in some embodiments, external controller 14 may include an additional RF or infrared interface for communication with other external devices, e.g., for transfer of urodynamic information.

External controller 14 may take the form of a portable, handheld device, like a pager or cell phone, that can be carried by patient 12. External controller 14 may include an internal antenna, an external antenna protruding from the recorder, or an external antenna that extends from the recorder on a cable and is attached to the body of patient 12 at a location proximate to the location of monitor 12 to improve wireless communication reliability. Also, in some embodiments, external controller 14 also may receive operational or status information from monitor 12, and may be configured to actively configure and interrogate the monitor to receive the information.

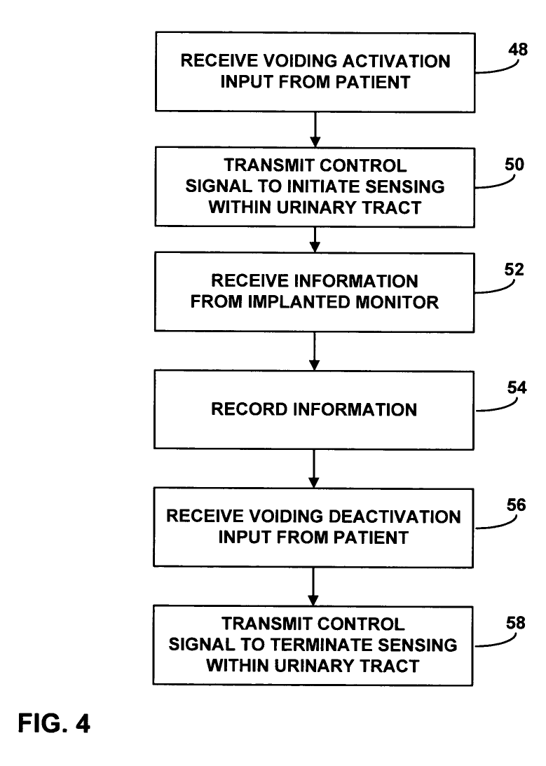

FIG. 4 is flow diagram illustrating operation of a urodynamic monitor 12 and external controller 14 in response to a voiding event activation command from a patient. As shown in FIG. 4, external controller 14 receives a voiding event activation command from patient 16 (48). In response, external controller 14 transmits a control signal to monitor 12 instructing the monitor to initiate sensing of one or more physiological parameters of the urinary tract (50). Implanted monitor 12 transmits urodynamic information, based on the sensed physiological parameters. External controller 14 receives the urodynamic information from implanted monitor 12 (52). External controller 14 then processes the information received from monitor 12, if necessary, and records the information in memory for evaluation by a physician or other care-giver (54).

Urodynamic monitor 12 may continue to collect urodynamic information for a specified period of time following receipt of the activation command. Upon expiration of the specified period of time, monitor 12 automatically terminates collection of urodynamic information. The specified time may be selected to correspond to the maximum expected duration of a voiding event, such as an attempt to void urine from the bladder, and may be on the order of several seconds to a few minutes.

Alternatively, as shown in FIG. 4, implanted monitor 12 may terminate collection of urodynamic information in response to a termination command received from external controller 14. For example, a patient may enter a voiding deactivation command into external controller 14 (56), e.g., at the end of a voiding event. In response, external controller 14 transmits a control signal to instruct monitor 12 to terminate sensing of urodynamic parameters (58).

During a voiding event, monitor 12 may intermittently or continuously transmit urodynamic information to external controller 14, e.g., in the form of measured parameters obtained at different sample times during the course of monitoring. Alternatively, monitor 12 may transmit information at the end of a monitoring period, either as measured parameters or processed values, such as average or trend data. Once the information is transmitted, monitor 12 waits for the next activation command for a subsequent voiding event.

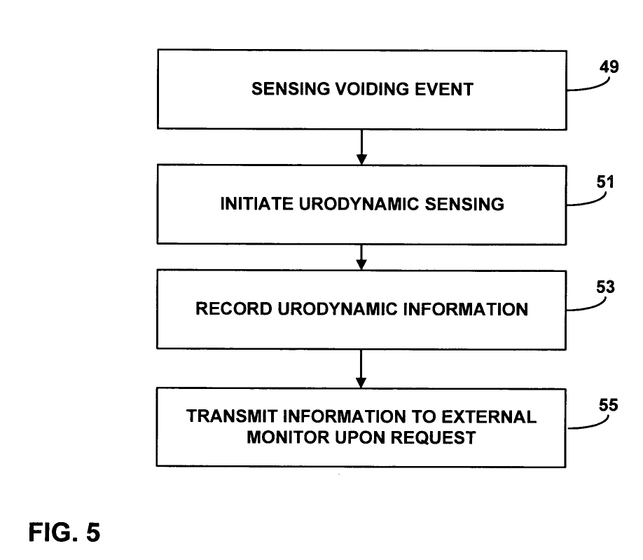

FIG. 5 is flow diagram illustrating operation of a urodynamic monitor 12 and external controller 14 in response to a sensed voiding event. In the example of FIG. 5, monitor 12 does not require a voiding event activation command from the patient 16. Instead, monitor is configured to automatically sense a voiding event (49), e.g., by detection of an elevation or change in pressure or urine flow. Upon automated detection of a voiding event, urodynamic monitor 12 initiates sensing of physiological parameters (51), and records urodynamic information based on the sensed parameters (53). The urodynamic information may be recorded within memory in monitor 12, and later transmitted to external controller 14 upon receipt of an interrogation request (55). Alternatively, the urodynamic information may be transmitted to external controller 14 as the information is obtained by monitor 12.

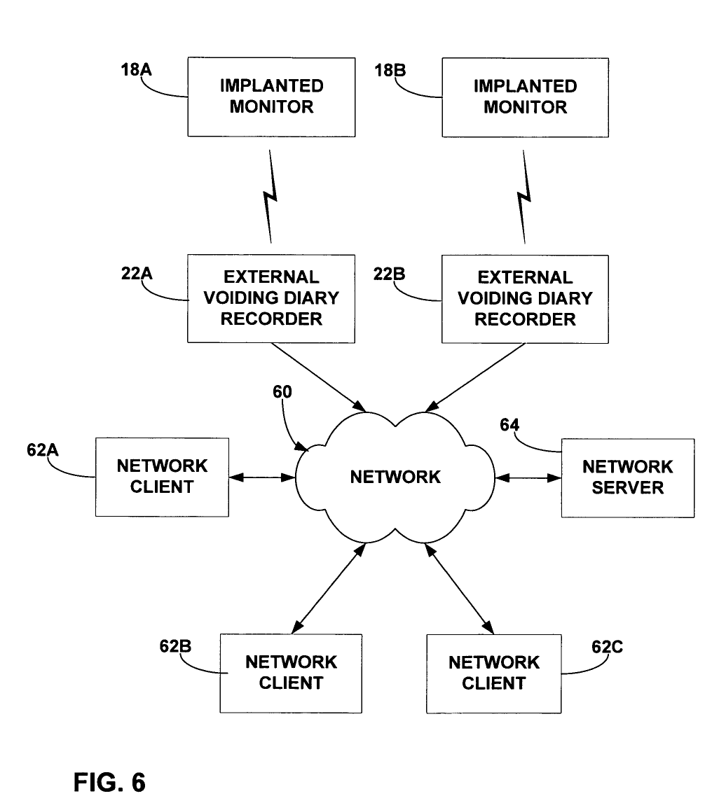

FIG. 6 is a functional block diagram illustrating a network 60 for communication of information obtained by one or more urodynanic monitors 12. Two implanted urodynamic monitors 12A, 12B, e.g., in different patients, are shown for purposes of illustration. However, information for any number of monitors 12 and patients 16 may be accessed via network 60. In particular, physicians or other medical personnel may view urodynamic information transmitted to external controllers 14A, 14B by implanted monitors 12A, 12B to evaluate urodynamic conditions. External controllers 14A, 14B are coupled to network 60 via wired or wireless connections, and transmit information obtained from monitors 12A, 12B to a network server 62 via the network.

Network server 62 may be equipped to analyze the information and generate appropriate reports or advisories for viewing by users via any of network clients 64A, 64B, 64C (collectively 64), coupled to network 60. For example, network server 62 may generate web pages or other output that conveys voiding diary information obtained by monitors 12A, 12B. Hence, network clients 64 may access information on network server 62 using web browsers. In this manner, one or more users, such as physicians, may remotely view voiding diaries for one or more patients 16. Network 60 may take the form of a local area, wide area or global computer network, such as the Internet.

In some embodiments, network server 62 may be configured to poll external controllers 14 to received information. Network server 62 also may be configured to transmit advisories by email, facsimile, text messaging, instant messaging or the like to network clients 64, when voiding diary results are available for a particular patient. In this manner, the user associated with a network client 64 is able to remotely monitor information concerning a patient's condition, as obtained by the implanted monitor 12, and act on that information, if appropriate.

The ability to formulate an automated voiding diary with a temporary or chronic implanted monitor 12, combined with remote monitoring capabilities, can support a wide range of patient management capabilities, tight control of drug management, disease diagnostics, and chronic disease management. In addition, the ability to formulate a voiding diary while that patient is at home and going about daily living activities can provide much more accurate and meaningful data. For example, a pressure monitor in the bladder may be used to monitor bladder function during voiding events occurring over a period of several days, and over the course of several activities such as rest, eating, drinking, and exercise.

Implanted monitor 12 also may be used chronically for management of spinal cord injury patients who do not have bladder sensation. Rather than timed, intermittent catheterization, implanted monitor 12 would allow a spinal cord injury patient to monitor fullness and void only when necessary. In this case, monitor 12 could transmit a signal to external controller 14 when a particular level of bladder fullness is reached, thereby triggering an alarm or advisory for voiding.

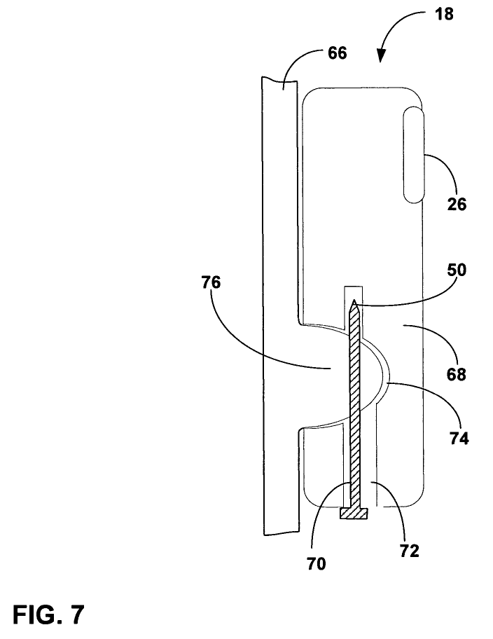

FIG. 7 is a cross-sectional side view of a urodynamic monitor 12 with a fixation structure. In the example of FIG. 7, monitor 12 is placed adjacent mucosal lining 66 within bladder 18 or urethra 20. Monitor 12 includes a capsule-like housing 68. A sensor 26 is exposed by housing 68 for interaction with the environment within bladder 18 or urethra 20. A shaft 70 extends through an internal channel 72 in the capsule-like housing 68 of monitor 12. Monitor 12 defines a vacuum cavity 74 on a side of the housing 68 adjacent mucosal lining 66. A vacuum port defined by channel 72 applies vacuum pressure to vacuum cavity 74 to draw a portion of mucosal tissue 76 into the cavity. The vacuum port is attached to a vacuum line (not shown) carried by an endoscopic delivery device. The vacuum line is coupled to an external vacuum source.

An elongated control rod (not shown in FIG. 7) may be applied via the endoscopic delivery device to drive shaft 70 into mucosal tissue 76. Shaft 70 may have a sharpened tip 77 that facilitates partial or complete penetration of tissue 76. Upon penetration of tissue 76 to secure monitor 12 relative to mucosal lining 66, vacuum pressure is deactivated and the endoscopic delivery device is withdrawn from urethra 20. Although shaft 70 is illustrated as penetrating tissue 76, in some embodiments, the shaft may be spring-biased to pinch a fold of the tissue and thereby secure monitor 12 at a desired position.

In some embodiments, shaft 70 may be manufactured from degradable materials that degrade over time, e.g., in the presence of urine, to release monitor 12 from mucosal lining 66. Alternatively, monitor 12 may release from mucosal lining 66 as mucosal tissue 76 sloughs away from mucosal lining 66. In either case, once the mucosal tissue 76 is released by shaft 70, monitor 12 detaches from mucosal lining 66 for passage through the urinary tract with urine flow or recovery with an endoscopic recovery device. Shaft 70, vacuum cavity 74 and the vacuum port defined by channel 72 form a fixation structure.

In general, monitor 12 may make use of fixation structures that are configured and function in a manner similar to any of the fixation structures disclosed in U.S. Pat. Nos. 6,285,897 and 6,698,056 to Kilcoyne et al. The Kilcoyne et al. patents provide examples of fixation mechanisms for attaching monitoring devices to the lining of the esophagus, including suitable degradable materials. The fixation structures described in the Kilcoyne et al. patents may be suitable for attachment of monitor 12 within bladder 18 or urethra 20. Examples include shafts, hooks, barbs, screws, sutures, clips, pincers, staples, tacks, and expandable frames. The contents of the Kilcoyne et al. patents are incorporated herein by reference in their entireties.

Although sensor 26 is depicted as having one or more surface components exposed to an environment within bladder 18 or urethra 20, in some embodiments, monitor 12 may include a hollow lumen to allow urine flow through the monitor. In this case, monitor 12 may have an annular cross-section, in a plane perpendicular to urine flow, and sensor 26 may be oriented such that sensor components are exposed to the interior of the hollow lumen. This type of configuration for monitor 12 may be particularly useful within urethra 16, and can be used to monitor flow rate, pressure, and timing of voiding, which may be advantageous in diagnosing benign prostate hyperplasia (BPH).

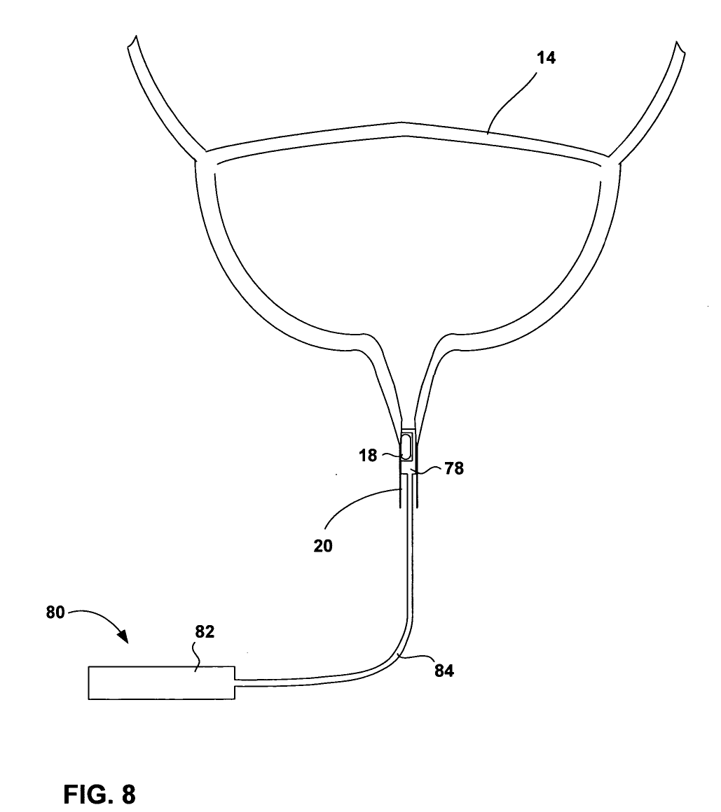

FIG. 8 is a schematic diagram illustrating deployment of a monitor 12 within a patient's urinary tract. As shown in FIG. 8, an endoscopic delivery device 80 serves to position and place monitor 12 within the urinary tract of patient 16. Delivery device 80 includes a proximal portion, referred to herein as a handle 82, and a flexible probe 84 that extends from handle 82 for insertion into urethra 20. Probe 84 is sized for passage through urethra 20 and may include a lubricating coating to facilitate passage.

Monitor 12 is coupled to a distal end 85 of delivery device 80 for delivery to a target location within the urinary tract. The target location may be within urethra 20 or within bladder 18. In some embodiments, delivery device 80 may include appropriate guidewires or other steering mechanisms to permit placement of monitor 12 on a lateral wall of bladder 18, as indicated by the position of monitor 12 depicted in FIG. 1.

Distal end 85 of delivery device 80 enters urethra 20 and extends into the urethra to the target location. The progress of distal end 85 may be monitored by endoscopic viewing or external viewing, e.g., with ultrasound or fluoroscopy. Monitor 12 is attached to the mucosal lining at the target location within bladder 18 or urethra 20, and the distal end 85 of delivery device 80 releases the monitor. Upon placement of monitor 12, flexible probe 84 and distal end 85 are withdrawn from urethra 20. Monitor 12 may be activated prior to placement within the urinary tract, or activated remotely by wireless communication or passage of a magnet in close proximity to monitor 12 to activate a switch carried by the monitor.

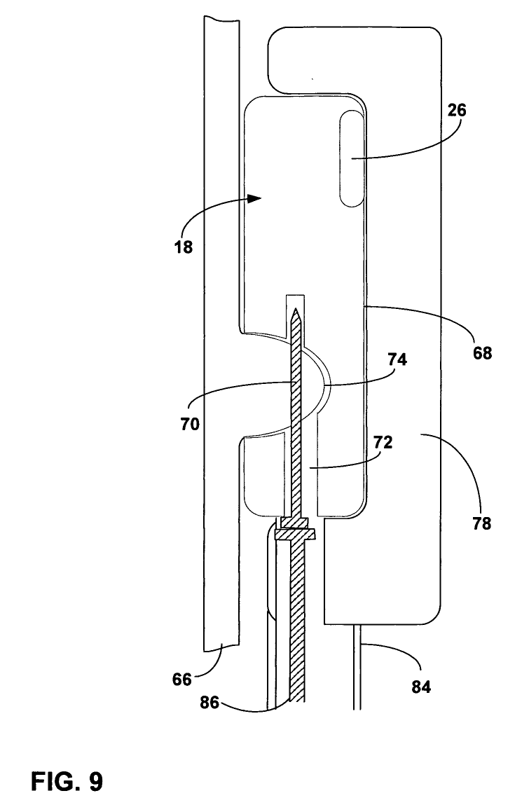

FIG. 9 is a cross-sectional side view illustrating positioning of monitor 12 of FIGS. 7 and 8 within distal end 85 of an endoscopic delivery device 80. As shown in FIG. 8, monitor 12 is held within a placement bay within distal end 85 of endoscopic delivery device 80. In this example, a physician advances elongated control rod 86 to drive shaft 70 into mucosal tissue 76. In general, elongated control rod 86 permits a physician to exert force to penetrate mucosal tissue 76. Elongated control rod 86 is flexible and extends though flexible probe 84 to handle 82 so that the physician can manipulate the elongated control rod. Before advancing elongated control rod 86, however, the physician activates a vacuum line to supply vacuum pressure to vacuum cavity 74 via channel 72 of monitor 12.

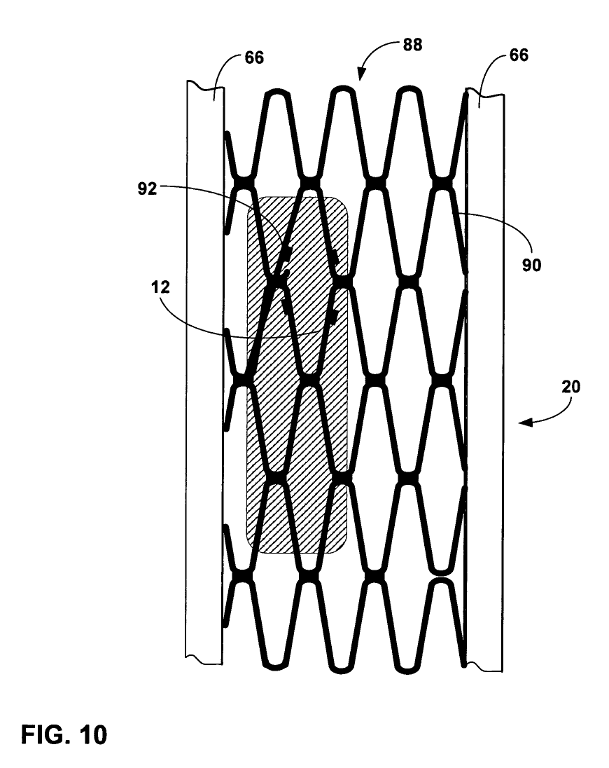

FIG. 10 is a side view of a monitor 12 with another fixation structure in the form of an expandable frame 88. As shown in FIG. 10, the capsule-like housing of monitor 12 has a diameter that is substantially less than the diameter of expandable frame 88 when the frame is in a fully expanded state. Upon expansion, frame 88 engages the mucosal lining of the interior wall 66 of urethra 20, much like a conventional stent used for restoring patency of blood vessels. In this manner, expandable frame 88 securely holds monitor 12 in place at a target location within the urethra 20. Details of a similar expandable frame and monitor arrangement are described in the aforementioned Christopherson et al. application.

The capsule-like housing of monitor 12 is attached to a portion of a wire grid 90 forming expandable frame 88. Monitor 12 may be welded, adhesively bonded, or crimped to one or more coupling points 92 on expandable frame 88. Wire grid 90 may take the form of a grid, network, or mesh of elastic wires that form a substantially cylindrical frame, similar to a conventional stent useful in restoring blood vessel patency. Examples of suitable materials for fabrication of wire grid 90 include stainless steel, titanium, nitinol, and polymeric filament, which can be absorbable or nonabsorbable in vivo, as described in the above-referenced Kilcoyne patents.

Expandable frame 88 may be intrinsically elastic such that it is self-expandable upon release from a restraint provided by an endoscopic delivery device. Alternatively, in some embodiments, a balloon or other actuation mechanism may be used to actively expand frame 88 to a desired diameter. In each case, expandable frame 88 extends radially outward to engage the wall of a urethra 20, and thereby place monitor 12 in contact with the lumen wall. In particular, upon expansion of frame 88, monitor 12 is placed within the lumen defined by urethra 20, and within the flow of urine through the urethra.

The position of monitor 12 within urethra 20 permits sensing of urodynamic parameters, such as pressure, flow rate, velocity, temperature, impedance, contractile force, and the like. In further embodiments, monitor 12 may include an ultrasonic imaging transducer to obtain snapshot ultrasound images of a region of interest during a voiding event. Monitor 12 senses the applicable physiological conditions and transmits information based on the sensed conditions to external controller 14. In some embodiments, expandable frame 88 may be electrically coupled to monitor 12 and form part of an antenna to facilitate reliable wireless telemetry.

Monitor 12 is depicted in FIG. 10 as being coupled to one side of expandable frame 88, and therefore resides adjacent a wall of urethra 20. In other embodiments, however, monitor 12 may be mounted to frame 88 such that monitor resides substantially centrally within urethra 16. For example, monitor 12 may be cantilevered or otherwise supported by frame 88 with expandable struts that place the monitor centrally within the aperture defined by the frame. In this case, monitor 12 may be constructed with a hollow lumen for passage of urine flow, and a sensor associated with monitor 12 may be oriented inward toward the lumen to sense conditions of the urine such as urodynamic conditions or urinalysis characteristics.

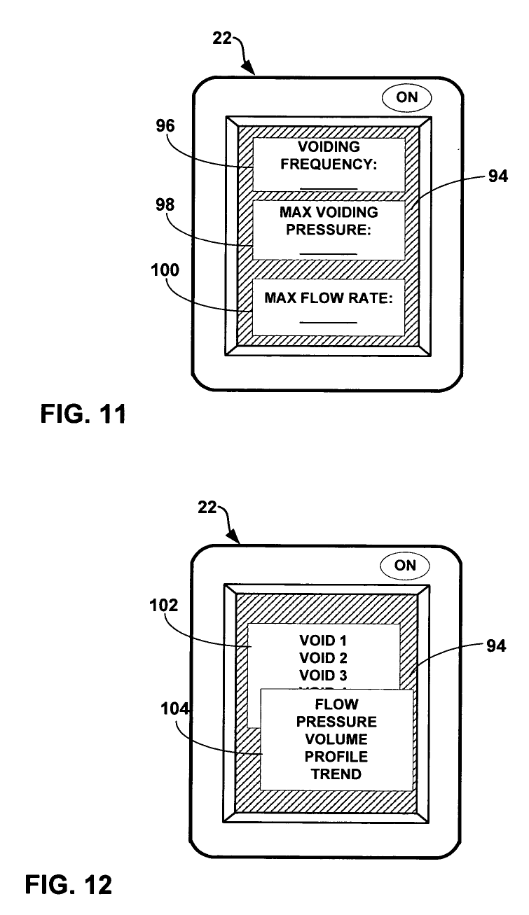

FIG. 11 is a conceptual diagram of an external controller 14 equipped to present voiding diary information to a user. External controller 14 includes a display screen 94 that presents urodynamic information associated with a particular voiding event. In the example of FIG. 11, display screen 94 presents the date and time 96 of the voiding event, a maximum voiding pressure 98 measured by monitor 12 during the course of the voiding event, and maximum flow rate 100 measured by monitor 12 during the course of a voiding event. A variety of additional urodynamic parameters may be presented on external controller 14, as well as average, maximum, minimum and trend data over a voiding event or over a series of voiding events. The parameters depicted in FIG. 11 are merely for purposes of illustration, and should not be considered limiting of the invention as broadly embodied herein.

FIG. 12 is another conceptual diagram of external controller 14, illustrating organization and selection of urodynamic information obtained from monitor 12. For example, external controller 14 may present a list 102 of voiding events throughout a period of time, such as a day or several days. If display screen 94 is equipped as a touchscreen display, or external controller 14 includes other input media, such as buttons, keys, or the like, a user may select one of the voiding events and access a set 104 of urodynamic information for the selected voiding event. The set 104 of urodynamic information, in turn, may permit the user to select individual parameters, as well as profile or trend data, for presentation on display screen 94. External controller 14 also may provide the capability to transmit the information to another device, e.g., via wireless or wired connection.

The preceding specific embodiments are illustrative of the practice of the invention. It is to be understood, therefore, that other expedients known to those skilled in the art or disclosed herein may be employed without departing from the invention or the scope of the claims. For example, the invention is not limited to deployment of a monitor at a particular location within the urinary tract. In various embodiments, a medical device may be located anywhere within the urinary tract where useful diagnostic information can be obtained.

In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

Many embodiments of the invention have been described. Various modifications may be made without departing from the scope of the claims. These and other embodiments are within the scope of the following claims.

1. A system for forming a urinary voiding diary for a patient, the system comprising:
an implantable urodynamic monitor to generate urodynamic information based on a sensed physiological condition within a urinary tract of a patient in response to a voiding event activation command; and
an external controller to receive the urodynamic information from the monitor via wireless communication, and record the urodynamic information to form a voiding diary.

2. The system of claim 1, wherein the monitor terminates generation of the urodynamic information in response to a voiding event deactivation command.

3. The system of claim 1, wherein the monitor terminates generation of the urodynamic information upon expiration of a period of time following the voiding event activation command.

4. The system of claim 1, wherein the external controller includes a user input device to enter the voiding event activation command, and a wireless telemetry interface to transmit the voiding event activation command to the monitor.

5. The system of claim 1, wherein the monitor includes a wireless telemetry interface to receive the voiding event activation command.

6. The system of claim 1, wherein the monitor includes a magnet detector to detect proximity of a magnet to the monitor as the voiding event activation command.

7. The system of claim 1, wherein the monitor generates the voiding event activation command in response to detection of the onset of a voiding event.

8. The system of claim 7, wherein the monitor includes a sensor to detect the onset of the voiding event.

9. The system of claim 7, wherein the voiding event corresponds to at least one of an attempt to void urine from a bladder of the patient, a feeling of discomfort in the urinary tract, and involuntary leakage of urine.

10. The system of claim 1, wherein the monitor includes a memory to record the urodynamic information.

11. The system of claim 1, wherein the monitor includes a sensor configured to sense the physiological condition, and wherein the physiological condition includes at least one of pressure, flow, velocity, temperature, contractile force, and impedance.

12. The system of claim 1, wherein the external controller includes a display to present the urodynamic information to a user.

13. The system of claim 1, wherein the monitor includes a fixation element to fix the monitor within the urinary tract of the patient.

14. The system of claim 1, wherein the implantable urodynamic monitor generates urodynamic information for a plurality of voiding events over a period of time in response to a plurality of voiding event activation commands, and the external controller records the urodynamic information to form a voiding diary for the plurality of voiding events.

15. The system of claim 1, further comprising a plurality of the implantable urodynamic monitors generating multiple sets of the urodynamic information, the external controller recording the multiple sets of urodynamic information to form the voiding diary.

16. A method for forming a urinary voiding diary for a patient, the method comprising:
controlling an implantable urodynamic monitor to generate urodynamic information based on a sensed physiological condition within a urinary tract of a patient during a voiding event; and
generating a voiding diary based on the urodynamic information.

17. The method of claim 16, further comprising transmitting the urodynamic information to an external controller via wireless communication, and recording the urodynamic information in memory in the external controller to form the voiding diary.

18. The method of claim 16, wherein controlling the monitor includes transmitting a voiding event activation command to the monitor.

19. The method of claim 18, further comprising controlling the monitor to terminate generation of the urodynamic information upon expiration of a period of time following the voiding event activation command.

20. The method of claim 18, further comprising transmitting a voiding event deactivation command to the monitor to cause the monitor to terminate generation of the urodynamic information.

21. The method of claim 16, wherein controlling the monitor further comprises controlling the monitor to generate urodynamic information in response to user input indicating onset of a voiding event.

22. The method of claim 16, wherein controlling the monitor further comprises controlling the monitor to generate urodynamic information in response to detection of proximity of a magnet to the monitor.

23. The method of claim 16, wherein controlling the monitor further comprises controlling the monitor to generate urodynamic information in response to detection of the onset of a voiding event.

24. The method of claim 23, wherein the monitor includes a sensor to detect the onset of the voiding event.

25. The method of claim 16, wherein the voiding event corresponds to at least one of an attempt to void urine from a bladder of the patient, a feeling of discomfort in the urinary tract, and involuntary leakage of urine.

26. The method of claim 16, further comprising obtaining the sensed physiological condition from a sensor that senses at least one of pressure, flow, velocity, temperature, contractile force, and impedance.

27. The method of claim 16, further comprising transmitting the urodynamic information to an external controller via a wireless interface, and presenting the urodynamic information to a user via a display associated with the external controller.

28. The method of claim 16, wherein controlling the monitor includes controlling the monitor to generate urodynamic information for a plurality of voiding events over a period of time, and forming the voiding diary includes forming the voiding diary using the urodynamic information generated for the plurality of voiding events.

29. The method of claim 16, further comprising controlling a plurality of implantable urodynamic monitors to generate multiple sets of the urodynamic information, and forming the voiding diary based on the multiple sets of urodynamic information.

30-42. (canceled)

43. An implantable urodynamic monitor comprising:
a sensor to sense a physiological condition within a urinary tract of a patient; and
a processor to generate urodynamic information based on the physiological condition in response to a voiding event activation command indicative of an onset of a voiding event.

44. The monitor of claim 43, further comprising a wireless telemetry interface to transmit the urodynamic information to an external controller for formulation of a voiding diary based on the urodynamic information.

45. The monitor of claim 43, further comprising a wireless telemetry interface to receive the voiding event activation command from an external controller operated by the patient.

46. The monitor of claim 43, wherein the processor terminates generation of the urodynamic information in response to a voiding event deactivation command received from a user.

47. The monitor of claim 43, wherein the processor terminates generation of the urodynamic information upon expiration of a period of time following the voiding event activation command.

48. The monitor of claim 43, wherein the monitor includes a magnet detector to detect proximity of a magnet to the monitor as the voiding event activation command.

49. The monitor of claim 43, wherein the processor generates the voiding event activation command in response to detection of the onset of a voiding event.

50. The monitor of claim 49, wherein the monitor includes a sensor to detect the onset of the voiding event.

51. The monitor of claim 43, wherein the voiding event corresponds to at least one of an attempt to void urine from a bladder of the patient, a feeling of discomfort in the urinary tract, and involuntary leakage of urine.

52. The monitor of claim 43, further comprising a memory to record the urodynamic information.

53. The monitor of claim 43, wherein the sensor is configured to sense at least one of pressure, flow, velocity, temperature, contractile force, and impedance.

54. The monitor of claim 43, wherein the monitor includes a fixation element to fix the monitor within the urinary tract of the patient.
